(19) United States
(12) Patent Application Publication
(10) Pub. No.: US 2008/0206741 A1
(43) Pub. Date: Aug. 28, 2008

(54) Virus Recovery Medium, Use Thereof and Viral Diagnostic Kit Including Same

(76) Inventor: Robert Alexander, Victoria (AU)

Correspondence Address: BROOKS KUSHMAN P.C., 1000 TOWN CENTER, TWENTY-SECOND FLOOR, SOUTHFIELD, MI, 48075, US

(21) Appl. No.: 11/813,910

(22) PCT Filed: Jan. 13, 2006
(86) PCT No.: PCT/AU06/00044

§ 371 Date: Jul. 13, 2007

(30) Foreign Application Priority Data
Jan. 14, 2005 (AU) 2005900169

(51) Int. Cl. C12Q 1/70; C12N 7/00

(52) U.S. Cl. 435/5; 435/239

(57) ABSTRACT

The present invention relates to a virus recovery medium and a viral diagnostic kit comprising the same. The virus recovery medium is supplemented with a hormone and an enzyme. The hormone is preferably a glucocorticoid hormone, more preferably dexamethasone. The enzyme is preferably a protease, more preferably trypsin.

The present invention relates to a virus recovery medium, its use and a viral diagnostic kit comprising same. More particularly, the invention relates to a virus recovery medium that is dosed with a hormone, such as dexamethasone and an enzyme, such as trypsin.

Conventional diagnostic procedures for identifying viruses include seeding containers with particular cell lines selected on their sensitivity to certain viruses and then inoculating the cell culture with a biological sample putatively containing a virus. Such biological samples include among other things saliva, urine, faeces, cerebrospinal fluid (CSF), respiratory fluids and swabs such as those from the mouth, nasal cavity, throat, skin and genitals. The inoculated cell culture is then incubated and the cells examined for cytopathic effects induced by the virus. As certain viruses only grow on certain cells, the virus can be identified on the basis of the cell type in which it either induces a cytopathic effect (CPE) or does not induce a cytopathic effect.

There are a number of alternative protocols to this procedure including removing cells which have been inoculated with a virus preparation and subjecting the cells to trypsinisation and detecting viruses by monoclonal antibodies specific for viral-derived polypeptides labelled with a reporter molecule such as a fluorescein (FITC) molecule. A further alternative is to include a cover slip within a culture tube in order to enhance recovery of the cells.

The conventional tube (or traditional-drum) method utilises screw cap tubes which are seeded with appropriate cell lines. After reaching about 80% of cell confluency the tube is inoculated with an appropriate specimen and monitored for CPE for up to three weeks. Daily monitoring of CPE is required for the first week. Less frequent monitoring is necessary for the second and third weeks. Often, blind passage is required to enhance virus recovery.

One of the disadvantages of the conventional tube method is that it is time and labor intensive because daily monitoring of the tubes is required. Generally, two people inspect the same tube for CPE by light microscope to avoid subjectivity. In addition, not all viruses cause a visible CPE and those which do not are unable to be detected by this method. Furthermore, CPE formation monitored in the conventional tube method is highly dependent on the sensitivity of the cell lines and the capability of the virus to produce visible CPE. Toxicity of the specimen may also disadvantageously produce changes similar to viral CPE giving a false result. Also, some viruses produce CPE only after a long period of time (for example, Cytomagalovirus (CMV)). Thus, as results obtained by the conventional tube method are predominantly based on CPE detection and are not routinely confirmed by any other method, inaccurate diagnosis can occur.

Using the conventional tube method it is also practically impossible to use more than 2 or 3 tubes per specimen due to the resulting accumulation of tubes (40 specimens per day creates 500 tubes in first week alone).

The shell vial method is currently the most advanced method utilised by those in the art for virus recovery. The shell vial method utilises a 5 ml plastic vial (16 mm in diameter) with a translucent lid. Following an appropriate treatment, a round (13 mm) coverslip is inserted into the vial. The vial is seeded with a sensitive cell line which grows a monolayer on the cover slip. When the monolayer reaches about 80-90% confluency, the medium is discarded and the vial is inoculated with the patient's specimen. Then, the incubated vial is monitored for CPE, followed by the removal of the cover slip. The slip can then be fixed to a microscope slide and stained by monoclonal antibodies.

The advantage of the shell vial method is that virus recovery can be enhanced by centrifugation of vials after inoculation which can shorten the length of time taken to obtain results to as little as 2-3 days. Further, using the shell vial method there is no need to wait for visible CPE. The cover slip can be removed on the second or third day and stained with appropriate monoclonal antibodies and results (specific CPE) confirmed by using antibody-antigen staining.

However, the shell vial method also has a number of disadvantages, it is time consuming as the cover slips require special treatment: multiple washings with detergent and acetone followed by washing in distilled water and sterilisation. The cover slips also have to be manually inserted into the vials.

Another disadvantage of the shell vial method is that like the conventional tube method daily observation for CPE is necessary. Further, if immunofluorescent staining is necessary, the procedure required is complicated and time consuming. The medium from the shell vial has to be discarded and the cover slip manually (using specific forceps) removed, air dried and fixed to a microscope slide (using vacuum grease). The removal of cover slips is tedious, since the cover slips may be broken by rough manipulation, or unintentionally turned and fixed to the microscope slide with the monolayer upside down. Another complication may arise due to the seeded cells also growing on the bottom of the cover slip thus fixing it to the vial. Removal of such cover slips is very laborious.

Practically, using the shell vial method it is impossible to use more than 2 or 3 tubes per specimen due to the accumulation of tubes (40 specimens per day creates 500 shell vials per week). Further, a large amount of monoclonal antibodies is required for immunofluorescent staining in order to cover the round 13 mm cover slip.

The 96 well plate method is another method which is used only in limited cases for recovery of viruses which grow on the same cell line (for example, if the wells are seeded with LLC-MK2 cell line recovery of parainfluenza and also influenza viruses is possible). Monoclonal antibodies are used for diagnosis in conjunction with a 96 well plate.

This method has advantages in that the samples are relatively easy to manipulate when seeded with a cell line; large numbers of specimens can be inoculated onto the same plate; enhancement by centrifugation is possible; only a small amount of media is required (only 0.2 mL instead of 1-1.5 mL used in shell vials); antigen-antibody techniques may be used for confirmation of results; and the method also enables easy to “read” monitoring of CPE.

However, the 96 well plate method requires the whole plate to be used for antigen-antibody detection which is not generally practical. Also, the entire plate has to be used on the same day, even when the number of specimens is smaller than required for the whole plate. This means that for each day a new set of different plates must be used. Further, commonly only one or two different cell lines can be used per plate (the same type of specimen is inoculated onto the plate). As such, methods confirming the detection of viruses have to be done on the whole plate and at the same time. This disadvantageously results in a situation where, once the detection is completed, there are no remaining cells available for a repeat procedure in case of an error or after a prolonged incubation period.

In the above methods, the cell culture media used is often dosed with additives to allow improved virus recovery. It has been found by the present inventor that a cell culture media which is dosed with both hormone and enzyme advantageously optimises virus recovery by maintaining cell line sensitivity at its maximum, as well as aiding in the attachment of viruses to the cell wall and in some cases reducing the time taken to obtain a result. This has led to the virus recovery medium of the invention that may advantageously be used in the recovery of all viruses suitable for cell culture as set out in the following description.

The present invention also aims to provide a kit that facilitates a rapid, efficient and inexpensive means for alleviating the disadvantages of the known micro titre tray assemblies and providing enhanced virus recovery, preferably which is easily modified depending on the particular diagnosis which is to be conducted. The invention also relates to a method of detecting a virus using the virus recovery media of the invention. Advantageously, the invention provides for flexibility of options in use which have been hitherto unknown.

Accordingly, the present invention provides a virus recovery medium including a cell culture medium supplemented with at least one hormone and at least one enzyme.

As used herein, the terms “virus recovery medium” or “virus recovery media” refer to a medium or media which is used for virus growth and isolation. For example, virus recovery medium includes maintenance medium.

The enzyme added to the cell culture medium is not particularly limited and a person skilled in the art could identify suitable enzymes. In some embodiments, the term “enzyme” refers to a proteolytic enzyme. In these embodiments, the enzyme is preferably a serine or aspartate protease. Exemplary enzymes include trypsin, chymotrypsin or pepsin. In preferred embodiments, the enzyme is trypsin.

The hormone added to the cell culture medium is not particularly limited and a person skilled in the art could identify suitable hormones. In some embodiments, the term “hormone” refers to corticosteroids, preferably, a glucocorticoid. More preferably, the hormone is selected from dexamethasone, hydrocortisone, cortisone acetate, prednisone, prednisolone, methylprednisolone, betamethasone, triamcinolone, beclomethasone, fludrocortisone acetate, deoxycorticosterone acetate (DOCA), and aldosterone. In a preferred embodiment, the hormone is dexamethasone. The hormone may be either synthetic or naturally occurring.

While the combination of hormone and enzyme is the most preferred embodiment, alternatively it has been found that DMSO (dimethylsulfoxide) and DEAE (dextran) may also be useful.

The amount of the enzyme added to the culture medium is preferably within the range 1-5 μg/ml, and preferably about 2.5 μg/ml. The concentration of hormone in the culture medium is preferably within the range 10⁻⁴M-10⁻⁶M and preferably about 10⁻⁵M. However, in the case of dexamethasone and trypsin, which are preferred, it has been found that a cell culture media supplemented with about 2.5 μg/ml trypsin and dexamethasone at a concentration of about 10⁻⁵M gives the optimum result.

Accordingly, a specific embodiment of the invention provides a virus recovery medium that includes a cell culture media supplemented with 2.5 μg/ml trypsin and dexamethasone at a concentration of 10⁻⁵M.

The cell culture media which may be used in accordance with the invention is not particularly limited. For example, these may include medium-199, DMEM, RPMI-1640 or MEM-EAGLE. As will be readily recognised, however there is a wide variety of different media which can support the growth of cells and which are readily available to the skilled artisan. However, according to a preferred embodiment the cell culture media is MEM-EAGLE.

The cell culture media may be supplemented with additives that support cell and virus growth and such additives are known to those skilled in the art. It is known that particular cell lines and/or viruses may require specific additives for optimal growth and viability. Exemplary additives include L-glutamine, amino acids, antibiotics, serum, Hanks balanced salts, sugars such as D-glucose, inorganic salts, vitamins, phenyl red, buffers such as HEPES and surfactants such as Tween 80.

The Virus recovery medium described above may be used in conventional diagnostic procedures for identifying viruses such as the conventional tube method and the shell vial method. Alternatively, the virus recovery medium may be used in the method described below.

According to another embodiment of the invention there is provided the use of the above described virus recovery medium in a method for detection of viruses.

Accordingly there is provided a method of detecting a virus comprising:
(i) providing a cell line suitable for virus inoculation;
(ii) specific pre-treatment of a specimen to obtain a sample that potentially contains a virus to be detected;
(iii) inoculating the cells with a sample that potentially contains a virus to be detected;
(iv) incubating the inoculated cells;
(v) replacing the sample media with a virus recovery media comprising a cell culture medium supplemented with at least one hormone and at least one enzyme;
(vi) incubating the sample from (iv); and
(vii) detecting the virus.

The cell line may be selected to be suitable for the growth and isolation of a particular virus. For example, the cell line LLC-MK2 is suitable for detection of parainfluenza viruses, MDCK for influenza viruses; HEP-2 for Respiratory syncitial virus (RSV) and MRC-5 for cytomegalovirus (CMV), herpes simplex virus (HSV), Enteroviruses and Rhinoviruses. A skilled artisan could select the appropriate cell line for the growth and isolation of a given virus.

As used herein, the term “a sample that potentially contains a virus to be detected” includes sample specimens obtained from subject that may or may not be infected with a virus. Therefore the sample may contain a detectable virus or may be virus free. Suitable samples may be obtained from saliva, serum, urine, faeces, cerebrospinal fluid (CSF), respiratory fluids such as bronchial alveolar lavages and nasopharyngeal aspirates, and swabs such as those from the mouth, nasal cavity, throat, skin and genitals. The sample specimen may be prepared for use by dilution with a suitable media that is compatible with the cell line and virus.

The subject may be any species of animal that may be infected with a virus. For example, the subject may be a bird, fish or mammal. In some embodiments, the subject is a mammal. Suitable mammals include farmed animals such as sheep, cattle, pigs, deer and the like, companion animals such as dogs, cats, rabbits, guinea pigs and the like, laboratory animals such as mice, rats, monkeys and the like, captive animals such as those kept in zoos and humans. Preferred mammals are humans. In other embodiments, the subject may be a bird, particularly farmed birds such as chickens and turkeys. Specific pre-treatment methods of specimens to obtain samples suitable for virus detection are well known by the skilled artisan but may include, without being limited to, sonication and centrifugation.

The virus recovery medium according to the invention may be used for the recovery of a number of different viruses which are suitable for cell culture. The skilled artisan will recognise that such viruses include, but are not limited to, the respiratory viruses, Parainfluenza 1, 2, 3, 4 (PI 1, 2, 3, 4), Influenza A,B (Inf A,B), Respiratory syncitial virus (RSV), Adenovirus (AD), Rhinovirus (RH), Cytomegalovirus (CMV), and viruses from the Enterovirus group (ENT) consisting of Echovirus, Coxakievirus, Enterovirus and Poliovirus, and also non-respiratory viruses such as, but not limited to, Herpes simplex virus (HSV) 1, 2, Varicella zoster virus (VZV), Rubella, mumps, measels, rotavirus and polyomavirus.

In the method of the invention the inoculated cells may be incubated with the sample using known conditions. For example, the inoculated cells may be incubated at 37° C. for a period that results in the infection of the cells with the virus, such as 45 to 90 minutes, especially 60 minutes. Incubation may be performed in an incubator or may be performed with centrifugation.

The virus may be detected using common detection methods known in the art such as immunodetection techniques such as immunofluorescence, staining, visualisation of CPE, commonly used molecular techniques such as polymerase chain reaction (PCR), reverse transcriptase PCR(RT-PCR) and nucleic acid sequence based amplification (NASBA).

The virus recovery medium according to the invention may advantageously be used relatively easily in a multi-well micro titre tray assembly. As will be readily recognised, however a wide variety of different cell culture tray assemblies exist, which are readily available to the skilled artisan. For example, microtitre plates containing multiple wells are well known in the art. In one embodiment, a tray assembly as described in Australian Innovation Patent No. 2001100242 may be used. Alternatively, and according to a preferred embodiment, the micro titre tray assembly includes first, second and third tray units each having a plurality of receptacles; and a plurality of sample wells which are complimentary with the receptacles, each of the wells being individually and separately retainable within and removable from a respective receptacle of the plurality of receptacles; wherein each of the second and third tray units is adapted to detachably engage the first tray unit enabling the assembly of a micro titre tray consisting of the first tray unit and the second tray unit and/or the third tray unit.

Each of the first, second and third tray units may take any suitable form, although it will be appreciated that rectangular tray units will generally be preferred. In order to provide various arrays of receptacles to receive sample wells with solution to be analysed, in a preferred embodiment the first tray unit includes an array of 48 receptacles in a 6×8 matrix, the second tray unit includes an array of 48 receptacles in a 6×8 matrix and the third tray unit includes an array of 16 receptacles in a 2×8 matrix. Accordingly, a double 6×8 (i.e. 12×8) micro titre tray can be formed using the first tray unit and the second tray unit, an 8×8 micro titre tray can be formed using the first tray unit and the third tray unit, and a 14×8 micro titre tray can be formed using all three tray units. It will be appreciated that additional interposed tray units may be included to extend the number of receptacles or wells as desired.

The attachment of the second and third tray units to the first tray unit may be achieved by any suitable means. For example, this may include a snap-fit engagement or the like. According to a preferred embodiment, the second and third tray units, when engaged with the first tray unit, abut opposing sides of the first tray unit. Preferably, the second and third tray units include peripheral arms that detachably engage complementary sleeves on either side of the first tray unit.

In a preferred embodiment, each of the wells is resiliently retainable in a respective receptacle. For example, each of the wells may be resiliently retainable within a respective receptacle by virtue of a friction fit. That is, each well may be tapered so that the base of the well may be inserted into a respective receptacle, but due to increase in diameter of the well, the well is lodged into its respective receptacle. Other alternatives will be readily determined by a person skilled in the art. For example, each of the wells may include at least one ridge on an external surface thereof which engages an internal wall, or internal walls of the respective receptacle of the first, second and/or third tray units causing the friction fit.

Preferably, at least one of the tray units is provided with identification means for identifying sample wells held in the receptacles. In particular, the identification means may include a reference grid wherein each row of the plurality of receptacles is provided with a corresponding letter code and each column of the plurality of receptacles is provided with a corresponding number code.

Each of the first, second and third tray units, or any combination thereof may be provided with a complimentary cover, preferably adapted to provide each of the sample wells with individual cover. In particular, the cover preferably includes a plurality of circular ridges each of which, when the cover is placed on the respective tray unit or units, encircles the opening of a respective sample well to substantially enclose a sample held in the well.

In another embodiment, the sample wells are not individually and separately retainable, but rather are retained as a small unit of sample wells. The units of sample wells are preferably only small, including up to four sample wells.

Accordingly, the micro titre tray assembly preferably includes first, second and third tray units each having a plurality of receptacles; and a plurality of sample well units, wherein each of the sample well units includes up to 4 sample wells each of which is complimentary with a respective receptacle, and wherein each of the sample well units is, as a unit, retainable within and removable from a number of receptacles of the plurality of receptacles corresponding to the number of sample wells of the sample well unit; wherein each of the second and third tray units is adapted to detachably engage the first tray unit enabling the assembly of a micro titre tray consisting of the first tray unit and the second tray unit and/or the third tray unit.

According to this embodiment, the sample well units including up to four sample wells may be configured depending on the particular diagnosis which is to be carried out. For example, it may be that it is desirable to present a sample in duplicate so that duplicate assays may be conducted. In certain embodiments, the tray units are configured such that the number of receptacles in each row or in each column of the assembly corresponds to the number of sample wells in each of the sample well units.

The sample well units may be manufactured as desired. In that regard, the sample wells of each of the sample well units may be integrally formed, or may be detachable from each other to form individual sample wells. In the latter of these two options, it should be understood that the connection of the individual sample wells may be achieved using any means available for detachably connecting small plastic articles to one another.

According to yet another aspect of the invention, there is provided a viral diagnostic kit comprising the virus recovery medium of the invention, a micro titre tray assembly as described above, and optionally forceps adapted to facilitate removal of the sample wells or sample well units from the micro titre plate.

The forceps may take any suitable form provided that secure grasping of the individual sample wells or a sample well units including up to four sample wells is facilitated. In a preferred embodiment the forceps are especially adapted for this purpose including a first portion to be inserted in the well and a second portion which cooperates with the first portion and grasps the outer surface of the well. The first portion is preferably circular in cross-section and of a size to provide only minimal clearance from the inner wall of the well when inserted in the well.

The above described assemblies and virus recovery medium may provide specific advantages over the known methods and assemblies currently available. In particular, the assemblies and solution may facilitate increased sensitivity, using five or more highly specific cell lines for the same specimen. Also, enhancement by centrifugation is available. In this regard, centrifugal forces enhance the viral absorption and thus shorten the time for viral detection, in some cases by up to 10 times. Additionally, between 8-16 specimens can be inoculated on the one plate resulting in more specimens being handled by a single operator. Still further, increased flexibility in design of the assemblies are provided by the first, second and third tray units.

Increased objectivity may be provided by methods employing the assemblies and solution using rapid detection technique by means of fluorescence or enzymatic labelling and producing confirmed results that are available (in 80% of common viruses) within 1-2 days. In this regard, the assemblies are versatile in that up to 14 different cell lines can be used allowing a large range of viruses to be detected using a combination of different monoclonal antibodies. CPE can also be monitored. Furthermore, since a specimen may be inoculated into different cell lines, different monoclonals can be used on the different cell lines and within a different time frame. In case of an error separate wells with inoculated specimen are still available to enable additional testing.

Methods incorporating the use of the assemblies and solution may also provide time saving benefits by allowing results to be available in 1-3 days. Such methods represent a large saving in hands-on work. The assemblies also enable cost effective testing as for example, only a small amount of monoclonal antibody is required for one test (20 μl compared to 60-80 μl of monoclonal antibody required for one test using the shell vial method).

In use, the plate is seeded with a plurality of cell lines. The choice of cell lines used for detection depending on the type of specimen and the presence of expected viruses. For example without limiting the invention to a particular cell line the LLC-MK2 cell line could be used for detection of Parainfluenza viruses; MDCK for Influenzae viruses; HEP-2 for RSV and MRC-5 cell line for the isolation of CMV, HSV, Enteroviruses and Rhinoviruses. Following seeding, each specimen is then inoculated onto a different row of the plate. For example, if the plate is 8×12, eight different specimens may be inoculated onto each plate, each specimen being inoculated into 12 wells of any given row on up to 12 different cell lines. This advantageously facilitates the detection of at least twelve viruses. It will be recognised, however, that this 96 well plate configuration can be changed according to specific diagnostic requirements. This simply requires an appropriate modification to the cell line selection to maintain specificity towards the viruses to be detected.

The present invention is further described by reference to the following non-limiting Figures and/or Examples.

Reference will now be made to the accompanying drawings which illustrate embodiments of trays useful with the virus recovery medium in the kit of one aspect of the invention in which:

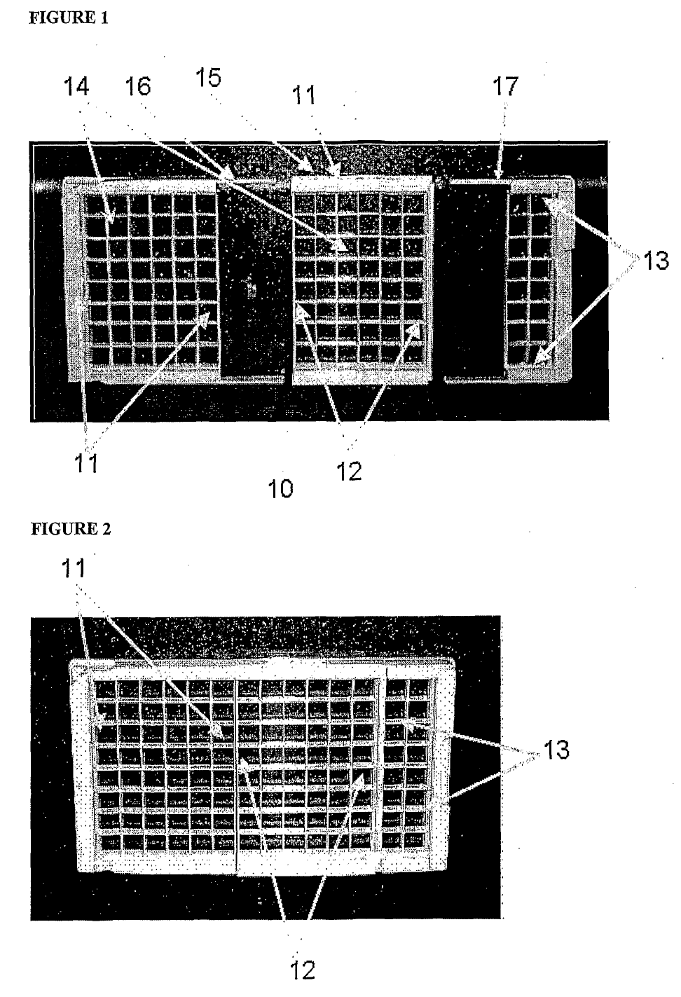

FIG. 1 illustrates the first, second and third tray units in a disassembled form;

FIG. 2 illustrates the first, second and third tray units in an assembled form;

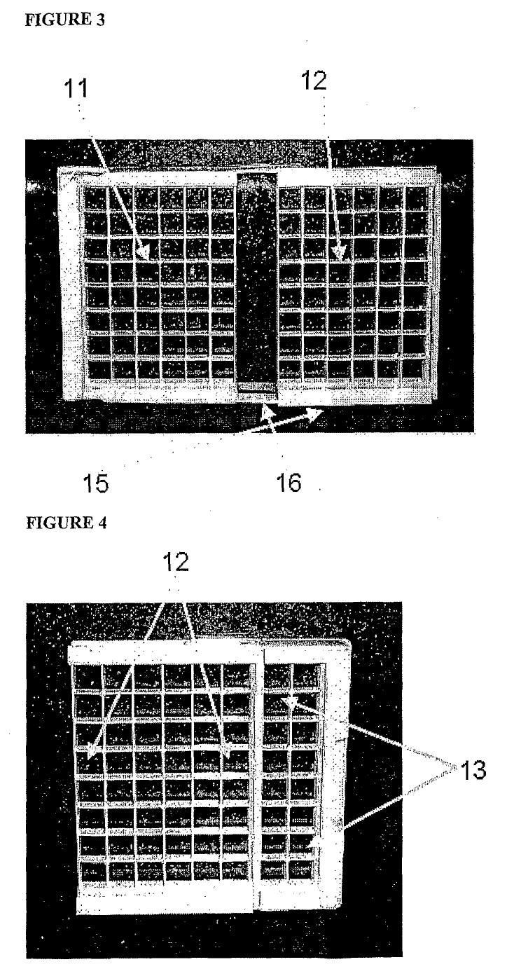

FIG. 3 illustrates the first and second tray units in a semi-assembled form;

FIG. 4 illustrates the first and third tray units in an assembled form;

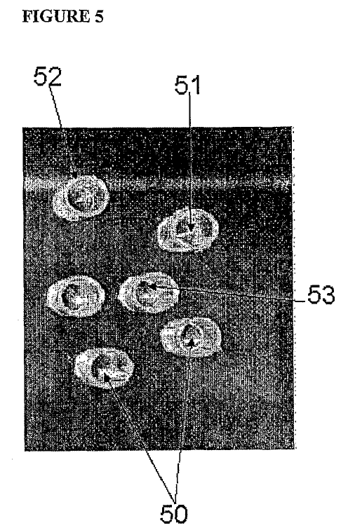

FIG. 5 illustrates a plurality of sample wells; and

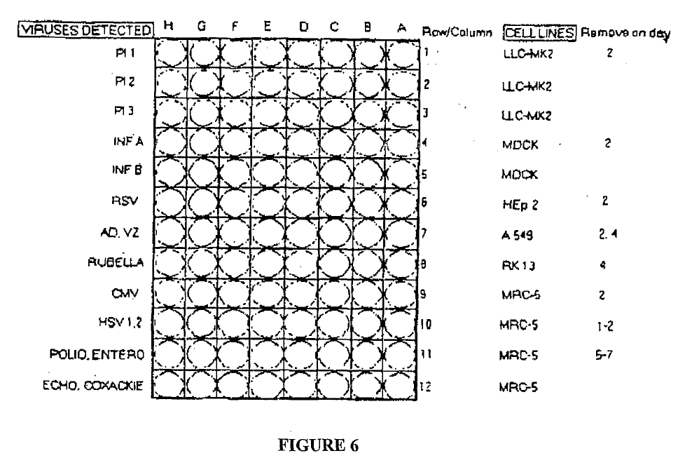

FIG. 6 illustrates a typical well configuration for a 12×8 matrix.

Referring to FIG. 1, a tray assembly 10 is provided that includes a first tray unit 11, a second tray unit 12 and a third tray unit 13. Each of the tray units 11, 12, and 13 includes a plurality of receptacles 14 that are adapted to receive individual sample wells (see FIG. 5) or sample well units that are formed from up to four of the individual sample wells.

The first tray unit 11 includes sleeves 15 along its outer edges. The sleeves 15 are adapted to receive peripheral arms 16 of the second tray unit on one side and peripheral arms 17 of the third tray unit on an opposing side. As such, the second and third tray units 12, 13 can be detachably engaged with the first tray unit 11 as best illustrated in FIG. 2 by sliding the peripheral arms 16, 17 into the sleeves 15 of the first tray unit 11. The engagement of the first and second tray units is particularly well illustrated in FIG. 3.

Referring to FIG. 5, each sample well 50 advantageously includes a tapered body 51, a base 52 and an annular lip 53 defining an opening to the well 50. With this configuration, each well can be resiliently retained within a respective receptacle of the first, second or third tray unit and removed as desired.

FIG. 6 exemplifies one example of a typical 12×8 well plate configuration (i.e. 2×6×8) including a listing of viruses to be detected, relevant cell lines and removal days for each line.

As will be seen from FIG. 6, the plate can be seeded with different cell lines in the following order:

Columns 1-3 LLC-MK2

Columns 4,5 MDCK

Column 6 Hep2

Column 7 A549

Column 8 RK13

Columns 9-12 MRC-5

A plate set up in this fashion would allow for example the selection of, without being limited to, the respiratory viruses Parainfluenza 1, 2, 3, 4 (PI 1, 2, 3, 4), Influenza A,B (Inf A,B) RSV, Adenovirus (AD), Rhinovirus (RH), Cytomegalovirus (CMV), and viruses from the Enterovirus group (ENT) consisting of Echovirus (Eco), Coxsackievirus (cox), Enterovirus (Ent) and Poliovirus (Polio), as well as, but not limited to, the non-respiratory viruses Herpes simplex virus (HSV) 1, 2, and Varicella zoster virus (VZV).

In a further example, configuration of a 6×8 well plate may consist of cell lines seeded in the following order:

| | |
|---|---|
| Columns 1-3 | A 549 |
| Columns 4-6 | MRC-5 |

This would allow for example the detection of viruses such as CMV, HSV 1, 2, VZV, AD, and those from the Enterovirus group.

Finally a configuration of 14×8 wells would ultimately allow for the detection of pathogens like PI 1, 2, 3, 4; Inf A,B; RSV; AD; RH; ENT (Echo, cox, Ent, Polio); HSV 1, 2; VZV; Rubella; Mumps; Measels; Rotavirus; Polyomavirus and also other pathogens-viruses using appropriate cell lines.

The removal of the cell lines, due to the individual nature of the wells, can be selective depending on the time schedule which is appropriate for the specific viral detection in question. In particular, if the detection of PI 1-4 is desired, taking row A for example, wells A 1 to A 3 are removed on day two. Similarly, if the detection of Inf A, B is desired, wells A 4 and A 5 are removed on day two. However, if the detection of Entero is required, then well A 11 is removed on the appropriate day (1-3). The specific nature of the individual wells facilitates this selective removal and viral detection.

Reference will now be made to a particular procedure which may be followed using the kit of one aspect of the invention, many steps of which may be optional and should not be considered to be limiting on the invention in any way.

Using vacuum and sterile glass pasteur pipettes, medium is aspirated from all wells to be inoculated. Disposal of pasteur pipettes in a large sharps container is advantageously facilitated.

Using a disposable pipette, an appropriate number of wells of the well plate are inoculated with approximately 150-200 μl specimen per well. The remaining specimen is stored at −70° C. The lid is then replaced on the plate and the date written over the wells inoculated in the plate.

The plate is then weighed on a digital balance and balanced with balance plates and cards until all plates are equivalent weights (+/−0.5 g) and can be balanced in a centrifuge. The centrifuge is run at about 37° C. and 3500 rpm for a period of 60 min. Using vacuum and sterile pasteur pipettes each specimen is then aspirated from each well, and using a fresh disposable pipette for each specimen, each well is filled with the virus recovery medium of the invention.

The specimens are then incubated in a humidified environment at 37° C. in a CO₂ incubator (5%) by carefully placing the plates in the CO₂ incubator and incubating at 37° C. for up to seven days after inoculation of the last specimen.

Immunofluorescent staining is advantageously used for detection of specific viruses in single wells, using specific monoclonal antibodies. Generally, the following procedure is followed: Using vacuum suction, the medium is removed from the appropriate well(s) and the wells removed from the plate using special forceps and transferred into a different holder. These are then air dried for 5 minutes. 300 μl of cold acetone is then added to each well and allowed to fix for 15 minutes at −20° C. The fixative is then discarded and the sample again air dried for 2-3 minutes. A specific monoclonal antibody (primary) is then added to each well and the cover plate put in place and the samples incubated for 30 minutes at 37° C. The samples are then removed from the incubator and each well filled with PBS. The PBS is then discarded. This process is repeated four more times. Again, the sample is air dried for 5 minutes, after which a secondary antibody is added to each well. Following this, incubation of the sample again takes place followed by repeated treatments with PBS as mentioned above and a final wash with double distilled water. A small amount (1 drop) of a specially prepared mounting medium is then added and the results observed under fluorescent microscope.

Throughout this specification and the claims which follow, unless the context requires otherwise, the word “comprise”, and variations such as “comprises” and “comprising”, will be understood to imply the inclusion of a stated integer or group of integers or steps but not the exclusion of any other integer or group of integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A virus recovery medium comprising a cell culture medium supplemented with at least one hormone and at least one enzyme.

2. A virus recovery medium according to claim 1 wherein the hormone is a glucocorticoid hormone.

3. A virus recovery medium according to claim 2 wherein the hormone is selected from dexamethasone, hydrocortisone, cortisone acetate, prednisone, prednisolone, methylprednisolone, betamethasone, triamcinolone, beclomethasone, fludrocortisone acetate, deoxycorticosterone acetate (DOCA), and aldosterone.

4. A virus recovery medium according to claim 3 wherein the hormone is dexamethasone.

5. A virus recovery medium according to claim 1 wherein the enzyme is a serine or aspartate protease.

6. A virus recovery medium according to claim 5 wherein the enzyme is selected from trypsin, chymotrypsin and pepsin.

7. A virus recovery medium according to claim 6 wherein the enzyme is trypsin.

8. A virus recovery medium according to claim 1 wherein the enzyme is present in an amount in the range of 1 to 5 μg/mL, especially about 2.5 μg/mL.

9. A virus recovery medium according to claim 1 wherein the hormone is present at a concentration of 10⁻⁴ to 10⁻⁶ M, especially about 10⁻⁵ M.

10. A method of detecting a virus comprising:
(i) providing a cell line suitable for virus inoculation;
(ii) specific pretreatment of a specimen to obtain a sample that potentially contains a virus to be detected;
(iii) inoculating the cells with a sample that potentially contains a virus to be detected;
(iv) incubating the inoculated cells;
(v) replacing the sample media with a virus recovery media comprising a cell culture medium supplemented with at least one hormone and at least one enzyme;
(vi) incubating the sample from (iv); and
(vii) detecting the virus.

11. A method according to claim 10 wherein the hormone is a glucocorticoid hormone.

12. A method according to claim 11 wherein the hormone is selected from dexamethasone, hydrocortisone, cortisone acetate, prednisone, prednisolone, methylprednisolone, betamethasone, triamcinolone, beclomethasone, fludrocortisone acetate, deoxycorticosterone acetate (DOCA), and aldosterone.

13. A method according to claim 12 wherein the hormone is dexamethasone.

14. A method according to claim 10 wherein the enzyme is a serine or aspartate protease.

15. A method according to claim 14 wherein the enzyme is selected from trypsin, chymotrypsin and pepsin.

16. A method according to claim 15 wherein the enzyme is trypsin.

17. A method according to claim 10 wherein the enzyme is present in an amount in the range of 1 to 5 μg/mL, especially about 2.5 μg/mL.

18. A method according to claim 10 wherein the hormone is present at a concentration of 10⁻⁴ to 10⁻⁶ M, especially about 10⁻⁵ M.

19. A viral diagnostic kit comprising:
(i) a virus recovery medium comprising a cell culture medium supplemented with at least one hormone and at least one enzyme; and
(ii) a micro titre tray assembly.

20. A viral diagnostic kit according to claim 19 wherein the micro titre tray assembly comprises multiple interconnected but detachable micro titre trays.
